(19) United States
(12) Patent Application Publication
(10) Pub. No.: US 2026/0199648 A1
(43) Pub. Date: Jul. 16, 2026

(54) Rectal Liquid Infusion System

(71) Applicant: Hayden Osborn, Bridgeport, CT (US)

(72) Inventor: Hayden Osborn, Bridgeport, CT (US)

(21) Appl. No.: 19/445,910

(22) Filed: Jan. 12, 2026

Related U.S. Application Data
(60) Provisional application No. 63/743,729, filed on Jan. 10, 2025.

(51) Int. Cl. A61M 31/00; A61M 39/10; A61M 39/28; A61M 25/10

(52) U.S. Cl. CPC A61M 31/00; A61M 39/10; A61M 39/28; A61M 2025/105

(57) ABSTRACT

A rectal liquid infusion system includes a rectal catheter assembly having a drug delivery connector, a supply assembly having an enteral connector, and an adapter having a drug delivery connector adapted to fluidly couple with the drug delivery connector of the rectal catheter assembly and an enteral connector adapted to fluidly couple with the enteral connector of the supply assembly, whereby the rectal catheter assembly and the supply assembly are in fluid communication via the adapter. The drug delivery connector of the rectal catheter assembly takes the form of a male Luer lock connector and the drug delivery connector of the adapter takes the form of a female Luer lock connector, while the enteral connector of the supply assembly takes the form of a female ISO 80369-3 compliant connector and the enteral connector of the adapter takes the form of a male ISO 80369-3 compliant connector.

### FIELD OF THE INVENTION

The present invention relates generally to a rectal liquid infusion system and more specifically to such a system employing an adapter configured to allow for communication between a standard enteral feeding system (such as those employing feeding bags and tubing sets that can be fed through a peristaltic pump or the like) and the port of a standard rectal tube/catheter.

### BACKGROUND OF THE INVENTION

As is known, rectal catheters are often inserted in patients in compromised states, such as those in intensive care units (ICUs). One of the benefits of such rectal tubes/catheters is that they may be used for the delivery of certain medications that are not appropriate for IV delivery, and when the medications cannot be ingested orally due to the compromised nature of the patient. Such rectal tubes/catheters include a syringe connection (typically a Luer lock connection) such that the medications can be delivered by syringe.

One such medication commonly delivered via rectal tube/catheter is lactulose, which is used as a first-line agent for the treatment of hepatic encephalopathy. Oral lactulose may be administered; however, research has shown that the pH of the stool decreases more rapidly (and there is a significantly decreased time in lowering ammonia levels) when administered rectally. The manufacturer’s recommendation for preparing lactulose for rectal administration is to mix 300 ml of lactulose solution in 700 ml of water or physiologic saline, and then to deliver the mixture rectally.

However, it must be recognized that the largest typical syringe that is often readily available in a hospital setting is 60 ml. Thus, in order to deliver the prepared 1000 ml lactulose mixture, it is necessary to refill and inject a 60 ml syringe more than 16 times. This is a time consuming and tiring process for caregivers.

It would be far more desirable if the prepared 1000 ml lactulose mixture could be provided in a feeding bag or the like, and then pumped into the rectal tube/catheter using a peristaltic pump or the like. However, there has heretofore been no satisfactory mechanism for allowing this to be done.

U.S. Patent Application Publication No. US 2024/0091432 A1 discloses a rectal catheter having two injection ports (34, 36) as well as a coupling port (40) configured to be fluidly coupled to a fluid bag (54), containing a fluid enema solution (56), via an extension tube (48). However, no infusion pump appears to be contemplated. Moreover, it appears that no adapter or the like is contemplated, such that the extension tube (48) is connectable to the port (40) by way of a compression tube lock (52) particularly tailored to mate with the extension tube (48). See, e.g., paragraph [0021]. Thus, there appears to be no disclosure, teaching or suggestion of allowing standard rectal catheters to be used with standard enteral feeding systems by way of an adapter or the like.

U.S. Patent Application Publication No. US 2022/0226560 A1 discloses a system for administering enemas and rectal irrigation having many similarities to US 2024/0091432 A1 described above, but also specifically contemplates the administering of lactulose (see paragraph ) via the system, as well as the optional employment of an irrigation pump (see paragraph ). However, as with US 2024/0091432 A1 described above, an overall system is disclosed, with there being no apparent disclosure, teaching or suggestion of allowing standard rectal catheters to be used with standard enteral feeding tubes/bags by way of an adapter or the like.

U.S. Patent Application Publication No. US 2019/0231969 A1 contemplates the use of an irrigation pump to infuse a wide variety of liquids, although lactulose is not specifically mentioned. See paragraph [0132]. Moreover, the use of a coupling component (15) between the injection liquid reservoir and the rectal catheter is shown and described. See, e.g., FIGS. 8-10 and paragraphs [0130]-[0131] and [0133]-[0134]. However, as discussed in paragraph [0131], it is contemplated that the coupling component (15) is employed to allow for the rectal catheter to be more universally used with irrigation liquid reservoirs of different manufacturers that employ different sized hoses. Thus, again, there does not appear to be any disclosure, teaching or suggestion of allowing standard rectal catheters to be used with standard enteral feeding tubes/bags by way of an adapter or the like.

In summary, all of the know prior art that contemplates the infusion of treatments via a rectal catheter contemplate entire systems. Thus, if the teachings of the prior art were to be employed, entirely new systems (typically, many of such systems) would be required to be purchased, inventoried and maintained by hospitals. Moreover, caregivers would need to be trained on how to use these new systems. Obviously, all of these considerations add complexity and cost to an already complex and costly health care system. It would be far more desirable if existing equipment already present in the hospital setting, and with which caregivers are already familiar, could be used more efficiently for the rectal infusion of medications and the like.

The present invention, therefore, aims to provide a mechanism by which standard enteral feeding systems (such as those employing feeding bags and tubing sets that can be fed through a peristaltic pump or the like) already present and in use in virtually all hospitals and patient care facilities can be connected to a port of a standard rectal tube/catheter, also already present and in use in virtually all hospitals and patient care facilities.

### SUMMARY OF THE INVENTION

In accordance with one aspect of the present invention, a rectal liquid infusion system includes a rectal catheter assembly including an insertion end adapted to be inserted in a rectum of a patient, the insertion end having a drug delivery orifice, and a proximal end including a drug delivery connector, the drug delivery connector and the drug delivery orifice being in fluid communication via a drug delivery channel. The system also includes a supply assembly including a reservoir adapted to contain a liquid to be infused and an enteral connector in fluid communication with the reservoir via a tubing set, the enteral connector of the supply assembly being incompatible with the drug delivery connector of the rectal catheter assembly. An adapter includes a first end having a drug delivery connector adapted to engage with the drug delivery connector of the rectal catheter assembly and a second end having an enteral connector adapted to engage with the enteral connector of the supply assembly, the drug delivery connector of the adapter and the enteral connector of the adapter being in fluid communication with one another via an internal passage. In this manner, the reservoir is fluidly communicable with the drug delivery orifice via the drug delivery channel of the rectal catheter assembly, the internal passage of the adapter and the tubing set of the supply assembly.

In some embodiments, the rectal catheter assembly further includes a balloon disposed adjacent to the insertion end, the balloon adapted to be inflated after the insertion end is inserted in the rectum of the patient in order to retain the insertion end in place. In certain of these embodiments, the rectal catheter assembly further includes an inflation port disposed at the proximal end thereof, wherein the inflation port is in fluid communication with the balloon so as to allow for inflation and deflation of the balloon with a fluid via the inflation port.

In some embodiments, the rectal catheter assembly further includes a collection vessel disposed adjacent to the proximal end thereof and a collection orifice disposed on the insertion end thereof, the collection vessel being in fluid communication with the collection orifice via a collection channel, whereby liquid or semi-liquid materials can be collected from the rectum of the patient. In certain of these embodiments, the rectal catheter assembly further includes a clamp disposed on the collection channel such that, when engaged, the clamp closes off the collection channel whereby collection of liquid or semi-liquid materials from the rectum of the patient is ceased.

In some embodiments, the drug delivery connector of the rectal catheter assembly and the drug delivery connector of the adapter comprise Luer lock connectors. In certain of these embodiments, the drug delivery connector of the rectal catheter assembly comprises a male Luer lock connector and the drug delivery connector of the adapter comprises a female Luer lock connector.

In some embodiments, the supply assembly further includes an infusion pump disposed between the reservoir and the enteral connector, the infusion pump configured to pump liquid contained in the reservoir through the enteral connector. In certain of these embodiments, the infusion pump comprises a peristaltic infusion pump. In certain embodiments, the tubing set of the supply assembly is removably insertable into the peristaltic infusion pump. In some embodiments, the reservoir of the supply assembly is adapted to contain at least 1000 ml of liquid.

In some embodiments, the enteral connector of the supply assembly and the enteral connector of the adapter comprise ISO 80369-3 compliant connectors. In certain of these embodiments, the enteral connector of the supply assembly comprises a female ISO 80369-3 compliant connector and the enteral connector of the adapter comprises a male ISO 80369-3 compliant connector.

In some embodiments, both the drug delivery connector of the rectal catheter assembly comprises a male Luer lock connector and the drug delivery connector of the adapter comprises a female Luer lock connector, and the enteral connector of the supply assembly comprises a female ISO 80369-3 compliant connector and the enteral connector of the adapter comprises a male ISO 80369-3 compliant connector.

In accordance with another aspect of the present invention, a rectal liquid infusion system includes a rectal catheter assembly including an insertion end adapted to be inserted in a rectum of a patient, the insertion end having a drug delivery orifice, and a proximal end having a drug delivery connector, wherein the drug delivery connector and the drug delivery orifice are in fluid communication via a drug delivery channel. A balloon is disposed adjacent to the insertion end, the balloon adapted to be inflated after the insertion end is inserted in the rectum of the patient in order to retain the insertion end in place, and an inflation port is disposed at the proximal end, wherein the inflation port is in fluid communication with the balloon so as to allow for inflation and deflation of the balloon with a fluid via the inflation port. The system also includes a supply assembly having a reservoir adapted to contain a liquid to be infused, an enteral connector in fluid communication with the reservoir via a tubing set, the enteral connector of the supply assembly being incompatible with the drug delivery connector of the rectal catheter assembly, and a peristaltic infusion pump disposed between the reservoir and the enteral connector, the peristaltic infusion pump configured to pump liquid contained in the reservoir through the enteral connector. An adapter includes a first end having a drug delivery connector adapted to engage with the drug delivery connector of the rectal catheter assembly and a second end having an enteral connector adapted to engage with the enteral connector of the supply assembly, the drug delivery connector of the adapter and the enteral connector of the adapter being in fluid communication with one another via an internal passage. In this manner, the reservoir is fluidly communicable with the drug delivery orifice via the drug delivery channel of the rectal catheter assembly, the internal passage of the adapter and the tubing set of the supply assembly. The drug delivery connector of the rectal catheter assembly comprises a male Luer lock connector and the drug delivery connector of the adapter comprises a female Luer lock connector, while the enteral connector of the supply assembly comprises a female ISO 80369-3 compliant connector and the enteral connector of the adapter comprises a male ISO 80369-3 compliant connector.

In some embodiments, the rectal catheter assembly further includes a collection vessel disposed adjacent to the proximal end thereof and a collection orifice disposed on the insertion end thereof, the collection vessel being in fluid communication with the collection orifice via a collection channel, whereby liquid or semi-liquid materials can be collected from the rectum of the patient. In certain of these embodiments, the rectal catheter assembly further includes a clamp disposed on the collection channel such that, when engaged, the clamp closes off the collection channel whereby collection of liquid or semi-liquid materials from the rectum of the patient is ceased.

In some embodiments, the tubing set of the supply assembly is removably insertable into the peristaltic infusion pump. In some embodiments, the reservoir of the supply assembly is adapted to contain at least 1000 ml of liquid.

In accordance with a further aspect of the present invention, a rectal liquid infusion system includes a rectal catheter assembly having a drug delivery connector, a supply assembly having an enteral connector, and an adapter having a drug delivery connector adapted to fluidly couple with the drug delivery connector of the rectal catheter assembly and an enteral connector adapted to fluidly couple with the enteral connector of the supply assembly, whereby the rectal catheter assembly and the supply assembly are in fluid communication via the adapter. The drug delivery connector of the rectal catheter assembly takes the form of a male Luer lock connector and the drug delivery connector of the adapter takes the form of a female Luer lock connector, while the enteral connector of the supply assembly takes the form of a female ISO 80369-3 compliant connector and the enteral connector of the adapter takes the form of a male ISO 80369-3 compliant connector.

Other features and advantages of the invention will become more apparent from consideration of the following drawings.

### BRIEF DESCRIPTION OF THE DRAWINGS

FIG. 1 is a schematic high-level view of a rectal liquid infusion system in accordance with an exemplary embodiment of the present invention;

FIG. 2 is schematic illustration showing in more detail a rectal catheter assembly portion of the rectal liquid infusion system of FIG. 1;

FIGS. 3A and 3B are schematic illustrations showing in more detail portions of the rectal catheter assembly of FIG. 2;

FIG. 4 is schematic illustration showing in more detail a liquid supply assembly portion of the rectal liquid infusion system of FIG. 1; and

FIG. 5 is schematic illustration showing in more detail an adapter portion of the rectal liquid infusion system of FIG. 1.

### DETAILED DESCRIPTION OF THE INVENTION

The present invention may be further understood with reference to the following description and the appended drawings, wherein like elements are referred to with the same reference numerals.

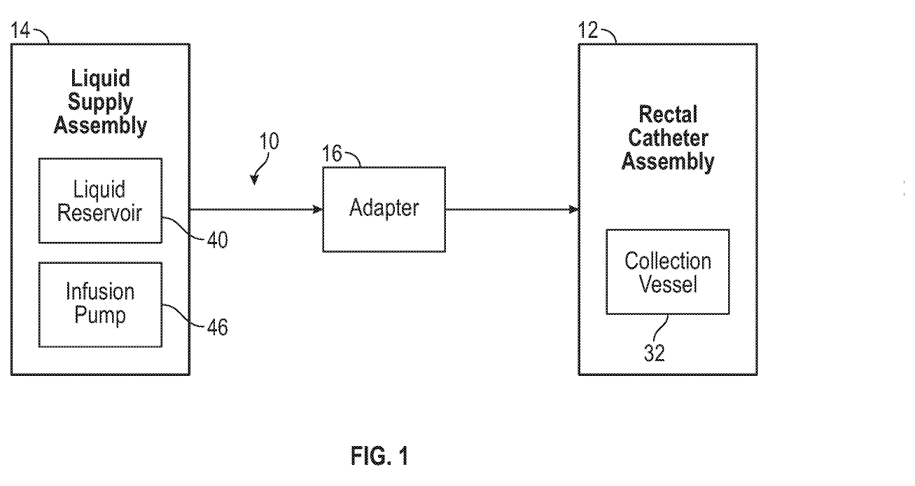

As shown very generally in FIG. 1, the rectal liquid infusion system (10) in accordance with the present invention generally includes three main components: a rectal catheter assembly (12), a liquid supply assembly (14) and an adapter (16), with the rectal catheter assembly (12) and the liquid supply assembly (14) being in fluid communication with one another via the adapter (16). Each of these components, along with their configuration and operation, will be described in more detail below.

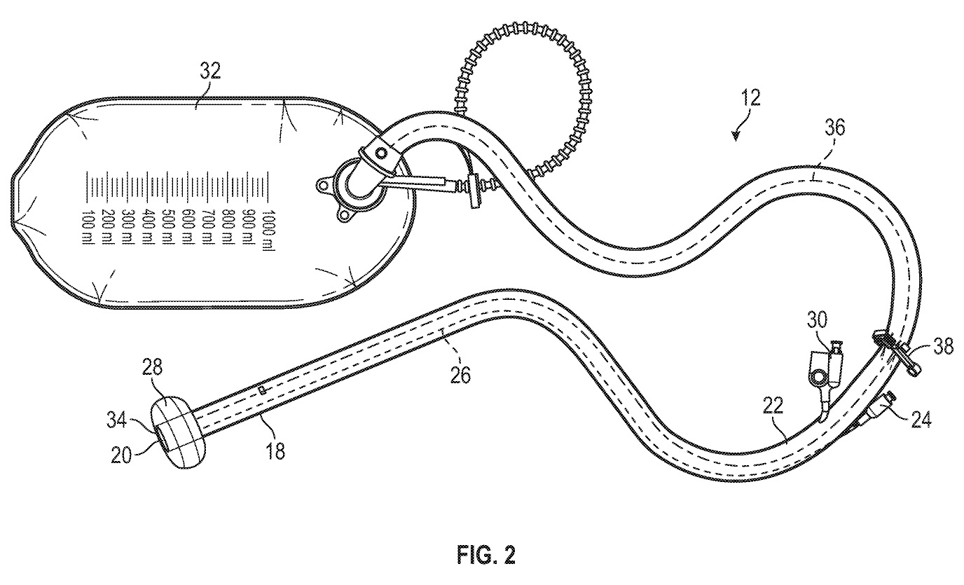

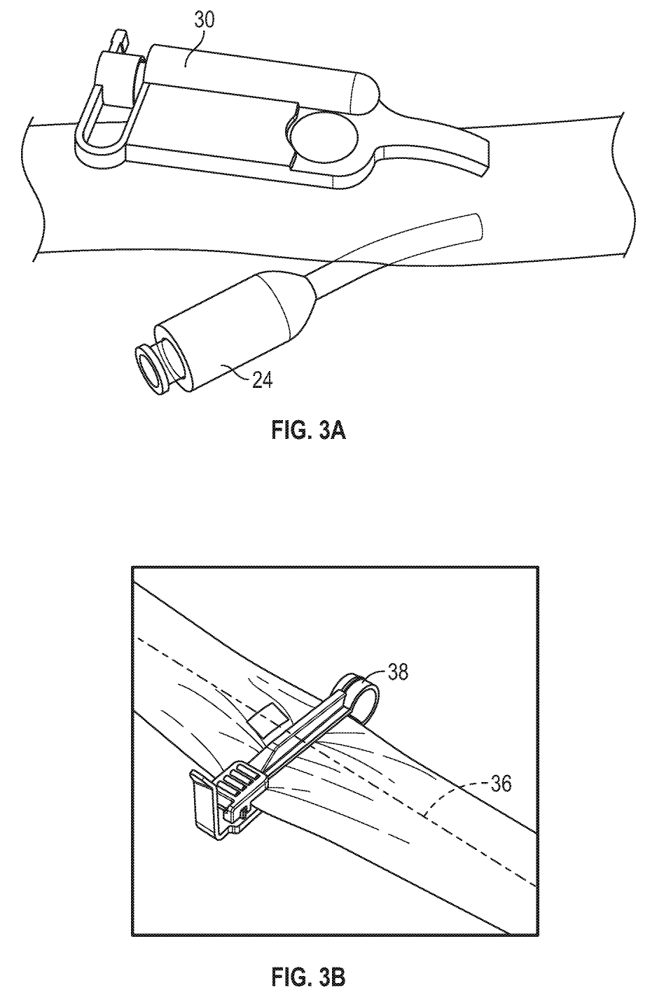

As best seen in FIGS. 2, 3A and 3B, the rectal catheter assembly (12) includes an insertion end (18) adapted to be inserted in a rectum of a patient (not shown), the insertion end (18) having a drug delivery orifice (20), and a proximal end (22) comprising a drug delivery connector (24). The drug delivery connector (24) and the drug delivery orifice (20) are in fluid communication via a drug delivery channel (26).

As is common, the rectal catheter assembly (12) also includes a balloon (28) disposed adjacent to the insertion end (18), the balloon (28) being adapted to be inflated after the insertion end (18) is inserted in the rectum of the patient (not shown) in order to retain the insertion end (18) in place. The balloon (28) may also seal the rectum when the rectal catheter assembly (12) is used as part of a fecal management system (FMS). As such FMS systems are well known and widely used, the operation thereof is not discussed in detail herein. An exemplary FMS is marketed by Convatec LTD. under the Flexi-Seal™ brand.

The rectal catheter assembly (12) also includes inflation port (30) disposed at the proximal end (22) thereof, wherein the inflation port (30) is in fluid communication with the balloon (28) so as to allow for inflation and deflation of the balloon (28) with a fluid via the inflation port (30). As is well-known, the fluid used to inflate the balloon may be a liquid, such as tap water, saline, etc., or it may be a gas, such as ambient air.

As noted above, the rectal catheter assembly (12) may comprise part of a fecal management system (FMS), in which case, the rectal catheter assembly (12) may also include a collection vessel (32) disposed adjacent to the proximal end (22) thereof and a collection orifice (34) disposed on the insertion end (18) thereof, the collection vessel (32) being in fluid communication with the collection orifice (34) via a collection channel (36), whereby liquid or semi-liquid materials can be collected from the rectum of the patient. A clamp (38) is disposed on the collection channel (36) such that, when clamped (as shown in FIG. 3B), the clamp (38) closes off the collection channel (36) whereby collection of liquid or semi-liquid materials from the rectum of the patient is ceased. The clamping of channel (36) to cease collection allows for the infusion of medications without their immediate evacuation from the rectum, such that a desired absorption time within the rectum may be achieved, before the clamp (36) is released and collection may re-commence. Again, as such systems are well-know, a more detailed description of the configuration and operation thereof is not presented herein.

As is common, the drug delivery connector (24) of the rectal catheter assembly (12) typically takes the form of a Luer lock connector. Even more specifically, the drug delivery connector (24) of the rectal catheter assembly (12) most often takes the form of a male Luer lock connector (as best seen in FIG. 3A). Again, this is typically the case with the Flexi-Seal™ brand FMS marketed by Convatec LTD, as well as many other rectal catheter assemblies typically stocked in hospitals and other patient care facilities.

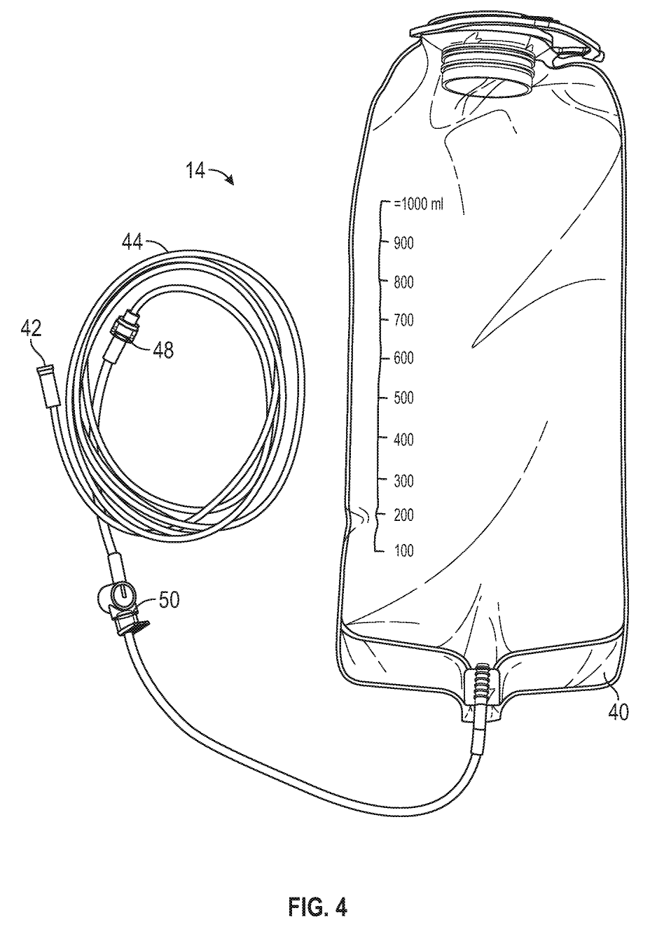

Turning now to FIG. 4 in addition to FIG. 1, the liquid supply assembly (14) is shown in greater detail. The liquid supply assembly (14) includes a reservoir (40) adapted to contain a liquid to be infused and an enteral connector (42) in fluid communication with the reservoir (40) via a tubing set (44). The liquid supply assembly (14) further includes an infusion pump (46) (schematically shown in FIG. 1) disposed between the reservoir (40) and the enteral connector (42), the infusion pump (46) being configured to pump liquid contained in the reservoir (40) through the enteral connector (42).

The reservoir (40) may take any of various forms, such as a flexible feeding bag, as are well known. Such bags are typically formed from BPA-free polyethylene (PE) or polypropylene (PP). The bag may be sized appropriately in consideration of the liquid to be infused. For example, as discussed above, when lactulose is to be administered, the manufacturer’s recommendation for preparing lactulose for rectal administration is to mix 300 ml of lactulose solution in 700 ml of water or physiologic saline, and then to deliver the mixture rectally. As such, the reservoir (40) is preferably adapted to contain at least 1000ml of liquid.

The infusion pump (46) may take the form of a peristaltic infusion pump, as are commonly known in the art. Most preferably, the tubing set (44) is removably insertable into the peristaltic infusion pump (46), and may be provided with various fittings (48, 50) that interface with the infusion pump (46). As such peristaltic infusion pump (46) and tubing set (44) configurations are extremely well known in the art, the configuration and operation thereof is not set forth in detail herein.

It should be noted that the enteral connector (42) is incompatible with the drug delivery connector (24) of the rectal catheter assembly (12). More specifically, while the drug delivery connector (24) of the rectal catheter assembly (12) takes the form of a Luer lock connector, the enteral connector (42) of the liquid supply assembly (14) takes the form of an ISO 80369-3 compliant connector (better known as ENFit® type connectors, ENFit® being a registered trademark of the Global Enteral Device Supplier Association (GEDSA)). Most preferably, the enteral connector (42) of the liquid supply assembly (14) takes the form of a female ISO 80369-3 compliant (i.e., ENFit® type) connector.

Exemplary liquid supply assemblies having the above characteristics (e.g., appropriately configured reservoir, tube set configured for use with peristaltic infusion pump, female ISO 80369-3 compliant (i.e., ENFit® type) connector, etc.) are marketed by Cardinal Health, Inc. under the Kangaroo™ brand, which, along with other appropriate assemblies, are typically stocked in hospitals and other patient care facilities.

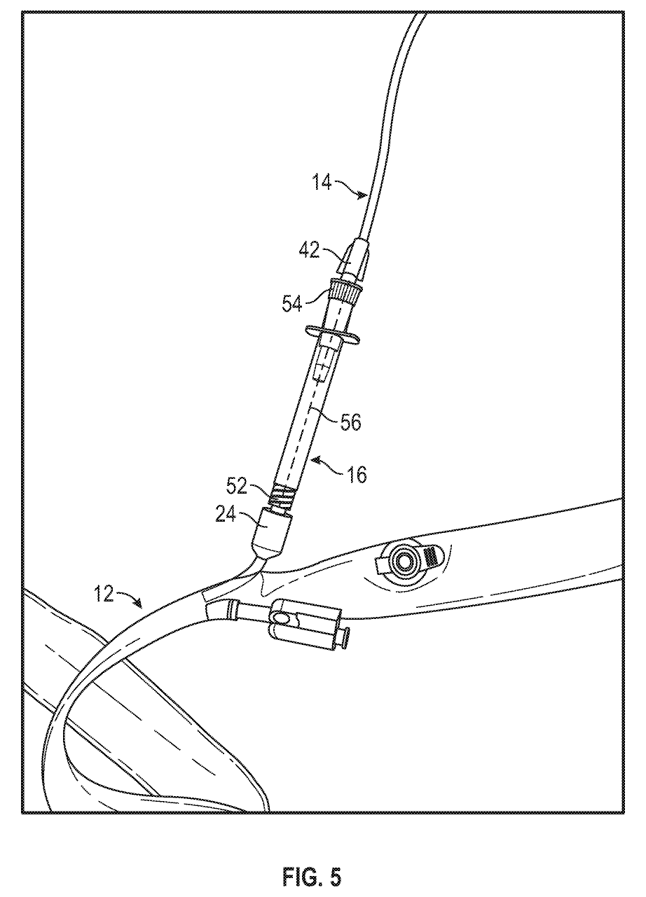

Turning now to FIG. 5 in addition to FIG. 1, the adapter (16) is shown in greater detail. The adapter has a first end comprising a drug delivery connector (52) adapted to engage with the drug delivery connector (24) of the rectal catheter assembly (12) and a second end comprising an enteral connector (54) adapted to engage with the enteral connector (42) of the liquid supply assembly (14), the drug delivery connector (52) of the adapter (16) and the enteral connector (54) of the adapter (16) being in fluid communication with one another via an internal passage (56). In this way, the reservoir (40) of the liquid supply assembly (14) is fluidly communicable with the drug delivery orifice (20) of the rectal catheter assembly (12) via the drug delivery channel (26) of the rectal catheter assembly (12), the internal passage (56) of the adapter (16) and the tubing set (44) of the liquid supply assembly (14).

As discussed above, the drug delivery connector (24) of the rectal catheter assembly (14) is preferably configured as a male Luer lock connector. As such, the drug delivery connector (52) of the adapter (16) takes the form of a female Luer lock connector. Similarly, as discussed above, the enteral connector (42) of the liquid supply assembly (14) preferably takes the form of a female ISO 80369-3 compliant (i.e., ENFit® type) connector. Consequently, the enteral connector (54) of the adapter (16) takes the form of a male ISO 80369-3 compliant (i.e., ENFit® type) connector. Thus, the adapter (16) is configured so as to have an internal passage (56) terminating at one and with a female Luer lock connector and at an opposite end with a male ISO 80369-3 compliant (i.e., ENFit® type) connector.

The adapter (16) is preferably formed from an inert material, such as a polymer -- with BPA-free polyethylene (PE) or polypropylene (PP) representing but two examples. Additionally, the adapter (16) may be formed as a single monolithic piece, or may be formed from multiple components joined together. Moreover, the adapter (16) may be formed using any of various techniques, such as molding (e.g., injection molding), machining or a combination thereof.

As should be recognized, the adapter (16) allows for standard enteral feeding systems (such as those employing feeding bags and tubing sets that can be fed through a peristaltic pump or the like) already present and in use in virtually all hospitals and patient care facilities to be connected to a port of a standard rectal tube/catheter, also already present and in use in virtually all hospitals and patient care facilities.

Although the invention has been described with reference to a particular arrangement of parts, features and the like, these are not intended to exhaust all possible arrangements or features, and indeed many other modifications and variations will be ascertainable to those of skill in the art.

What is claimed is:

1. A rectal liquid infusion system comprising:
a rectal catheter assembly comprising an insertion end adapted to be inserted in a rectum of a patient, the insertion end having a drug delivery orifice, and a proximal end comprising a drug delivery connector, wherein the drug delivery connector and the drug delivery orifice are in fluid communication via a drug delivery channel;
a supply assembly comprising a reservoir adapted to contain a liquid to be infused and an enteral connector in fluid communication with the reservoir via a tubing set, the enteral connector of said supply assembly being incompatible with the drug delivery connector of said rectal catheter assembly;
an adapter comprising a first end comprising a drug delivery connector adapted to engage with the drug delivery connector of said rectal catheter assembly and a second end comprising an enteral connector adapted to engage with the enteral connector of said supply assembly, the drug delivery connector of the adapter and the enteral connector of the adapter being in fluid communication with one another via an internal passage;
whereby the reservoir is fluidly communicable with the drug delivery orifice via the drug delivery channel of said rectal catheter assembly, the internal passage of said adapter and the tubing set of said supply assembly.

2. The rectal liquid infusion system of claim 1 wherein said rectal catheter assembly further comprises a balloon disposed adjacent to the insertion end, the balloon adapted to be inflated after the insertion end is inserted in the rectum of the patient in order to retain the insertion end in place.

3. The rectal liquid infusion system of claim 2 wherein said rectal catheter assembly further comprises an inflation port disposed at the proximal end thereof, wherein the inflation port is in fluid communication with the balloon so as to allow for inflation and deflation of the balloon with a fluid via the inflation port.

4. The rectal liquid infusion system of claim 1 wherein said rectal catheter assembly further comprises a collection vessel disposed adjacent to the proximal end thereof and a collection orifice disposed on the insertion end thereof, the collection vessel being in fluid communication with the collection orifice via a collection channel, whereby liquid or semi-liquid materials can be collected from the rectum of the patient.

5. The rectal liquid infusion system of claim 4 wherein said rectal catheter assembly further comprises a clamp disposed on the collection channel such that, when engaged, the clamp closes off the collection channel whereby collection of liquid or semi-liquid materials from the rectum of the patient is ceased.

6. The rectal liquid infusion system of claim 1 wherein the drug delivery connector of said rectal catheter assembly and the drug delivery connector of said adapter comprise Luer lock connectors.

7. The rectal liquid infusion system of claim 6 wherein the drug delivery connector of said rectal catheter assembly comprises a male Luer lock connector and the drug delivery connector of said adapter comprises a female Luer lock connector.

8. The rectal liquid infusion system of claim 1 wherein said supply assembly further comprises an infusion pump disposed between the reservoir and the enteral connector, the infusion pump configured to pump liquid contained in the reservoir through the enteral connector.

9. The rectal liquid infusion system of claim 8 wherein the infusion pump comprises a peristaltic infusion pump.

10. The rectal liquid infusion system of claim 9 wherein the tubing set of said supply assembly is removably insertable into the peristaltic infusion pump.

11. The rectal liquid infusion system of claim 1 wherein the reservoir of said supply assembly is adapted to contain at least 1000 ml of liquid.

12. The rectal liquid infusion system of claim 1 wherein the enteral connector of said supply assembly and the enteral connector of said adapter comprise ISO 80369-3 compliant connectors.

13. The rectal liquid infusion system of claim 12 wherein the enteral connector of said supply assembly comprises a female ISO 80369-3 compliant connector and the enteral connector of the adapter comprises a male ISO 80369-3 compliant connector.

14. The rectal liquid infusion system of claim 1 wherein:
the drug delivery connector of said rectal catheter assembly comprises a male Luer lock connector and the drug delivery connector of said adapter comprises a female Luer lock connector; and
the enteral connector of said supply assembly comprises a female ISO 80369-3 compliant connector and the enteral connector of the adapter comprises a male ISO 80369-3 compliant connector.

15. A rectal liquid infusion system comprising:
a rectal catheter assembly comprising:
an insertion end adapted to be inserted in a rectum of a patient, the insertion end having a drug delivery orifice,
a proximal end comprising a drug delivery connector, wherein the drug delivery connector and the drug delivery orifice are in fluid communication via a drug delivery channel,
a balloon disposed adjacent to the insertion end, the balloon adapted to be inflated after the insertion end is inserted in the rectum of the patient in order to retain the insertion end in place, and
an inflation port disposed at the proximal end, wherein the inflation port is in fluid communication with the balloon so as to allow for inflation and deflation of the balloon with a fluid via the inflation port;
a supply assembly comprising:
a reservoir adapted to contain a liquid to be infused,
an enteral connector in fluid communication with the reservoir via a tubing set, the enteral connector of said supply assembly being incompatible with the drug delivery connector of said rectal catheter assembly, and
a peristaltic infusion pump disposed between the reservoir and the enteral connector, the peristaltic infusion pump configured to pump liquid contained in the reservoir through the enteral connector; and
an adapter comprising a first end comprising a drug delivery connector adapted to engage with the drug delivery connector of said rectal catheter assembly and a second end comprising an enteral connector adapted to engage with the enteral connector of said supply assembly, the drug delivery connector of the adapter and the enteral connector of the adapter being in fluid communication with one another via an internal passage;
whereby the reservoir is fluidly communicable with the drug delivery orifice via the drug delivery channel of said rectal catheter assembly, the internal passage of said adapter and the tubing set of said supply assembly;
wherein the drug delivery connector of said rectal catheter assembly comprises a male Luer lock connector and the drug delivery connector of said adapter comprises a female Luer lock connector; and
wherein the enteral connector of said supply assembly comprises a female ISO 80369-3 compliant connector and the enteral connector of the adapter comprises a male ISO 80369-3 compliant connector.

16. The rectal liquid infusion system of claim 15
wherein said rectal catheter assembly further comprises a collection vessel disposed adjacent to the proximal end thereof and a collection orifice disposed on the insertion end thereof, the collection vessel being in fluid communication with the collection orifice via a collection channel, whereby liquid or semi-liquid materials can be collected from the rectum of the patient.

17. The rectal liquid infusion system of claim 16
wherein said rectal catheter assembly further comprises a clamp disposed on the collection channel such that, when engaged, the clamp closes off the collection channel whereby collection of liquid or semi-liquid materials from the rectum of the patient is ceased.

18. The rectal liquid infusion system of claim 15 wherein the tubing set of said supply assembly is removably insertable into the peristaltic infusion pump.

19. The rectal liquid infusion system of claim 15 wherein the reservoir of said supply assembly is adapted to contain at least 1000 ml of liquid.

20. A rectal liquid infusion system comprising:
a rectal catheter assembly comprising a drug delivery connector;
a supply assembly comprising an enteral connector;
an adapter comprising a drug delivery connector adapted to fluidly couple with the drug delivery connector of said rectal catheter assembly and an enteral connector adapted to fluidly couple with the enteral connector of said supply assembly, whereby said rectal catheter assembly and said supply assembly are in fluid communication via said adapter;
wherein the drug delivery connector of said rectal catheter assembly comprises a male Luer lock connector and the drug delivery connector of said adapter comprises a female Luer lock connector; and
wherein the enteral connector of said supply assembly comprises a female ISO 80369-3 compliant connector and the enteral connector of the adapter comprises a male ISO 80369-3 compliant connector.
